(19) United States
(12) Patent Application Publication
(10) Pub. No.: US 2003/0139672 A1
(43) Pub. Date: Jul. 24, 2003

(54) Apparatus and methods for analysing epithelial tissue histology

(76) Inventors: Michael Roger Cane, Hertfordshire (GB); Symon D?apos;oyly Cotton, Cambridgeshire (GB); Matthew Alexander Schumann, Cambridgeshire (GB); Michael Andrew Beadman, Hertfordshire (GB); Thomas Scott Carter, Cambridge (GB); Philip James Churchill White, Hertfordshire (GB)

Correspondence Address: RICHARD M. GOLDBERG, 25 EAST SALEM SREEET, SUITE 419, HACKENSACK, NJ, 07601, US

(21) Appl. No.: 10/240,071

(22) PCT Filed: May 8, 2001
(86) PCT No.: PCT/GB01/01986

(51) Int. Cl. A61B 6/00

(52) U.S. Cl. 600/473

(57) ABSTRACT

The present invention relates to apparatus and methodology for determining the distribution and concentration of chromophores within the epithelial tissue by illuminating an area of an epithelial tissue surface with visible and infrared spectral light. The epithelial tissue is illuminated with light of selected wavelength at a first intensity and an image of light remitted from the epithelial tissue surface is detected. The tissue is then illuminated at a second intensity and a second image is detected. A combined image is then produced such that each point of the image corresponds to a value of the percentage remittance at that position and has an intensity within that of the usable dynamic range of the detector.

### FIELD OF THE INVENTION

The present invention relates to an apparatus and methodology for the determination of the distribution and concentration of chromophores within an epithelial tissue.

### BACKGROUND OF THE INVENTION

The provision of histological information about epithelial tissue surfaces is particularly valuable for the determination and evaluation of abnormalities in such tissues.

An example of such an epithelial tissue is human and animal skin. The skin is composed of a series of layers of which the principal layers are the reticular dermis, papillary dermis, dermal-epidermal junction, epidermis and the stratum corneum (see FIG. 1). Each layer may be further subdivided into a series of sub-layers with more subtle physiological distinctions.

Dealing with each of the principal layers in turn, the reticular dermis layer forms the boundary between the skin and the subcutaneous tissue. The reticular dermis is primarily composed of a dense matrix of collagen fibres which is interspersed with elastic fibres. The papillary dermis is a highly vascular layer of the skin comprising the capillaries, which constitute the blood supply to the skin, and contacts the reticular dermis on the opposite side to the subcutaneous tissue. Collagen is also present in the papillary dermis as a more diffuse matrix of fibres compared to that of the reticular dermis.

In contact with the papillary dermis is a discreet thin layer of cells known as the dermal-epidermal junction. The cells which constitute this layer are rapidly dividing and continuously form epithelial cells and melanocytes which reside in the epidermis layer. Epithelial cells slowly migrate towards the external surface of the skin by displacement with recently formed epithelial cells below. As the cells progress towards the external surface of the skin, cell death and keratinisation occurs which ultimately gives rise to the external surface of the skin, known as the stratum corneum. The stratum corneum has the appearance of a series of scales or flakes which are continuously shed from the surface of the skin and replaced by more recently formed keratinised epithelial cells from below.

Conventional methods for the diagnosis of skin ailments involve the examination of the surface characteristics of a skin lesion. In addition, and dependant on the skin condition, a proportion or entire area of a skin lesion may be surgical excised and used for histological examination under a microscope.

There are a variety of skin conditions were the provision of histological information rapidly would be a valuable adjunct to enable the efficient diagnosis of a skin ailment. In the example of malignant melanoma, histological information could be vital to determining the prognosis of the disease. For instance, the ingression of melanocytes into the papillary dermis and in particular the depth of ingression has been correlated to the prognosis of the disease (Neville, C. D. “Melanoma: Issues of Importance to the clinician”, British Journal of Hospital Medicine, 1995). For this reason, a device that could provide histological information about an area of skin rapidly and by a non-invasive technique would be a distinct advantage.

The principal chromophores located in the skin include melanin, haemoglobin, oxy-haemoglobin and collagen. In normal healthy skin, melanin is located exclusively in the epidermis, and haemoglobin and oxyhaemoglobin are located primarily in the papillary dermis and to a lesser extent the reticular dermis. Collagen is located throughout the dermis, with the highest concentration residing in the reticular dermis. Abnormalities in the distribution of such chromophores can provide valuable information about the histology of a skin ailment and can be obtained by detecting and interpreting the distribution of different chromophores within the skin. In addition, the distribution of chromophores within any epithelial tissue may be analysed to provide information about any lesions of the tissue.

The present invention is concerned with the analysis of the spectra of light remitted from an epithelial surface following illumination. By illumination we mean the provision of an incident light of broad spectral composition incorporating, in particular, visible and infrared wavelengths of light.

The present invention is also directed to the determination of the spectral characteristics of the light remitted from the skin surface. By spectral characteristics we mean the intensity of specific wavelengths and wavelength ranges of the light remitted from the skin. Our application WO 98/22023 discloses a non-invasive method by which the skin colour co-ordinates and the papillary dermis thickness are determined by the analysis of light remitted from an area of skin following illumination.

Our co-pending United Kingdom patent application numbers 99 12 908 and 99 25 414 relate to advances and improvements in the determination of the concentration and distribution of chromophores within the skin. In particular, United Kingdom patent application number 99 12 908 relates to methods and apparatus by which the histology of the skin may be determined and the identification of the presence depth and concentration of chromophores within the skin. United Kingdom patent application number 99 25 414 relates to a method and apparatus for providing the information of the skin structure, more particularly, to mapping the surface of dermal papillae.

An object of the present invention is to provide a device for the measurement of epithelial tissue histology and enable the determination of the distribution and concentration of chromophores within the skin using conventional components and means for detection of the remitted light.

### BRIEF SUMMARY OF THE INVENTION

According to an aspect of the present invention there is provided an apparatus configured to create one or more spectral images of an epithelial surface for determination of the concentration and distribution of chromophores within the epithelial tissue surface comprising

a light source to illuminate an area of the epithelial tissue surface with visible and infrared spectral light;

wavelength selection means for selecting the wavelength of light remitted from the epithelial tissue surface;

intensity selection means for selecting the intensity of light remitted from one or more points of the epithelial tissue surface, and

detection means for detecting the intensity of remitted light at one or more points from the epithelial tissue surface to create an image such that each point of the image corresponds to a value of the percentage of incident light remitted.

By epithelial tissue we include the skin and the linings of the respiratory and digestive tracks, the retina or any other surface that may be accessed by non-invasive means. By non-invasive means, we mean that the epithelial tissue can be analysed in situ without the need for surgical excision of the tissue from a subject.

According to a further aspect of the invention there is provided a method of determining the concentration and distribution of chromophores within an epithelial tissue using a detector having a defined usable dynamic range, wherein said method comprises the steps of calibrating the intensity of light source and the homogeneity of the incident illumination illuminating the epithelial tissue surface at a first intensity of illumination; detecting the intensity of light remitted from the epithelial tissue surface; and illuminating the epithelial tissue with at least one further level of illumination and detecting the image such that substantially each point of the image is detected within the dynamic range of the detector.

According to a further aspect of the present invention there is provided a method of creating a combined image having each point of the image detected within the usable dynamic range of the detector wherein said method comprises the steps of: calibrating the intensity of light source and the homogeneity of the incident illumination; illuminating the epithelial tissue surface at a first intensity of illumination; detecting the intensity of light remitted from the epithelial tissue surface; and illuminating the epithelial tissue with at least one further level of illumination different from said first level detecting said image and, for each point of said image, selecting said first or second illumination intensity such that substantially each point of the image is detected within the dynamic range of the detector.

### BRIEF DESCRIPTION OF THE DRAWINGS

The invention will now be described, by way of example only, with reference to the accompanying drawings, in which:

FIG. 1 is a schematic representation of the structure of human skin;

FIG. 2 is a perspective view of the external appearance of one example of equipment incorporating the invention;

FIG. 3 shows the equipment of FIG. 2 in use;

FIG. 4 is a schematic representation of the arrangement of the components of the equipment of FIG. 2;

FIG. 5 is a side elevational view of the light generating subassembly which is housed in the equipment of FIG. 2;

FIG. 6 is a sectional view taken along the line Z-Z of FIG. 5;

FIG. 7 is a flow chart indicating the operational sequence of the invention;

FIG. 8 is a graph demonstrating the dependency of the intensity of the remitted light on the concentration of the chromophores within the skin;

FIG. 9 is a view of the remitted light image illustrating the area of exposed skin surrounded by intensity calibration patches and a corresponding calibration graph showing the intensity across the image;

FIG. 10 is an image of a malignant melanoma obtained by the equipment of FIG. 2 indicating the presence of the area of the image where melanin is present in the dermis;

FIG. 11 is an image of the haemoglobin concentration about a malignant melanoma obtained by the equipment of FIG. 2;

FIG. 12 is an image of the topology of the dermal-epithelial junction obtained by the equipment of FIG. 2; and

FIG. 13 is a flow chart illustrating the conventional approach to the diagnosis of skin ailments and the modified approach possible by using the apparatus of the present invention.

In the drawings the same reference numerals are used for like or corresponding parts in each of the Figures.

### BEST MODE FOR CARRYING OUT THE INVENTION

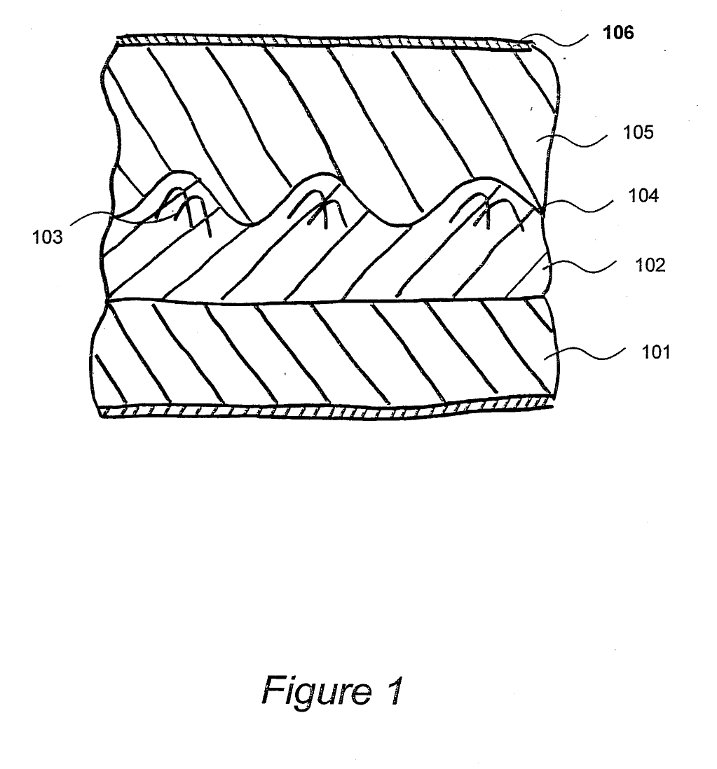

FIG. 1

The structure of human skin is shown schematically in FIG. 1. Normal human skin is composed of reticular dermis 101 which is composed of a dense network of collagen and elastic fibres. The papillary dermis 102 is a vascular tissue which comprises the capillary blood supply 103 to the skin and a more diffuse matrix of collagen fibres. The epidermis 105 is composed of epithelial cells and melanocytes constantly formed by cell division in the dermal-epidermal juncture 104. The external layer of the skin is termed the stratum corneum 106 and consists of keratin fibres and dead epithelial cells.

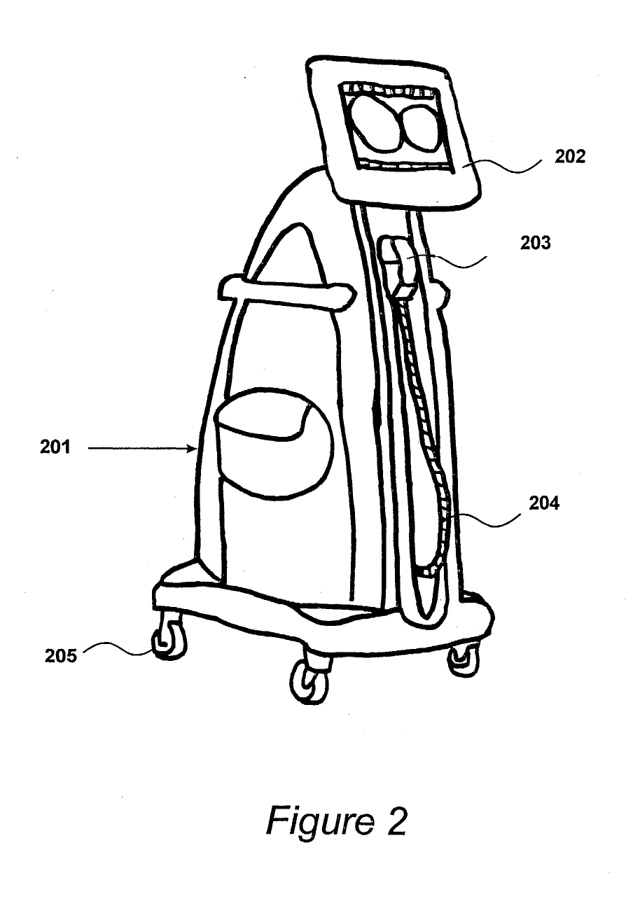

FIG. 2

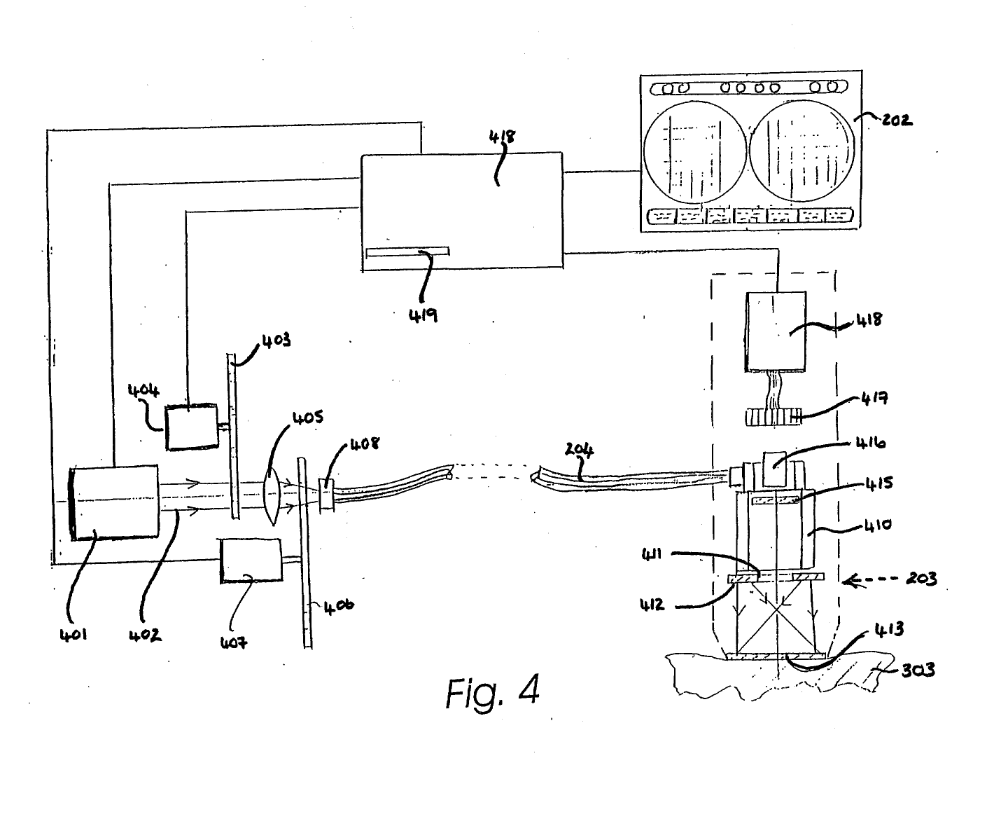

An example of the equipment of the invention is illustrated in FIG. 2. The equipment has a housing 201 which incorporates the system illustrated and discussed later in reference to FIG. 4. A display screen 202 with a touch screen operation is mounted on the housing 201. A light gun 203 in the form of a hand held “gun” is stored on the housing 201 when not in use, as indicated in FIG. 2. The “gun” 203 is connected to the internal system of the equipment by a flexible metal tubing 204 which contains a bundle of optical fibres, as is described in more detail later with respect to FIG. 4. The device is supported by castors 205, which enable the equipment of the invention to be conveniently moved into a required location.

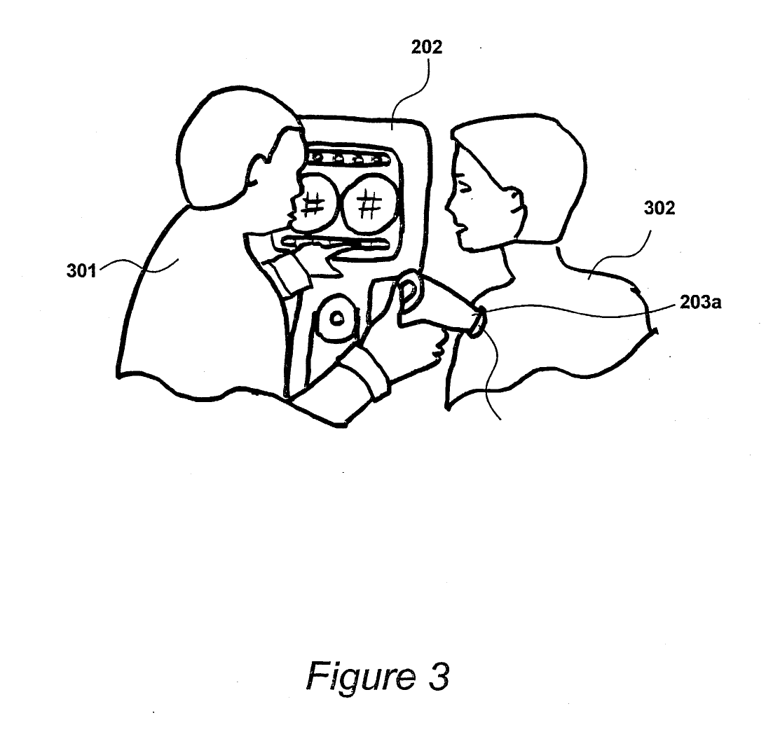

FIG. 3

In use an operator 301 removes the “gun” 203 from its stored position in the housing 201 and holds the free end 203a of the gun 203 against the target area 303 of the skin of a patient 302, as shown in FIG. 3. The operator 301 may then select options from the touch-screen 304 to initiate the illumination and imaging of the skin area.

The images obtained are displayed in a variety of formats on the display screen and the operator 301 can select specific representations and view the presence of specific chromophore constituents of the skin by selecting options from the display screen 202. The images are interpreted by a suitably trained operator and differences in the distribution of chromophores between the image obtained and the predetermined models of normal healthy skin can be visualised.

In the examination of skin ailments, a print out of the image or a digital image may be presented to a clinician to assist in the diagnosis of specific skin ailments.

Following the imaging of the skin, the gun 203 is replaced within the housing and a printout of the images obtained for recording purposes.

FIG. 4

FIG. 4 illustrates schematically the system which is contained in or mounted on the housing of FIG. 2. Illumination is provided by a light source 401 which is capable of providing a spectral range of illumination. The preferred light source is a xenon arc bulb capable of providing high intensity visible and infrared light illumination.

Light emitted by the source along the light path 402 is incident on a rotatable shutter disc 403, which comprises a series of apertures of varying dimensions to enable the selection of the intensity of the light from the source that is transmitted further along the device to illuminate the skin. The shutter disc is rotated by a stepper motor 404 to enable the alignment of the appropriate dimensioned aperture with the light path 402. The stepper motor is controlled by the computer 418.

A lens 405 enables the light transmitted through the aperture of the shutter disc to be focused onto one end 408 of the optical fibre bundle 204. Situated between the lens 405 and the optical fibre bundle 408 is a filter disc 406. The filter disc 406 comprises a series of filters arranged in a circle about the disc's central axis of rotation. The selected filter is rotated into the light path 402 by means of a further stepper motor 407 to select specific wavelengths of illumination incident on the skin.

Light of a selected intensity and wavelength is transmitted along the optical fibre bundle 204 to the gun 203. The gun 203 comprises a light tube 410 which is cylindrical with an internally reflective surface. The optical fibres 204 are arranged such that the termini of the fibres are located in a ring arrangement. This arrangement has been found to be beneficial for providing an homogenous intensity of illumination incident across the skin. In the preferred embodiment, the terminus of each optical fibre is equally spaced apart, although alternative arrangements such as arranging the termini of the fibres into discreet bundles which are located in a ring arrangement would also produce the desired illumination effect. There is a central aperture in the ring arrangement through which light remitted from the skin 303 can access a CCD (Charge Coupled Detector) 417.

Light emitted from the termini of the optical fibres passes through a first polarisation filter arrangement 412 of corresponding shape to the ring arrangement of the optical fibres. Polarised incident illumination light illuminates the target area 303 of the patient's skin through a transparent plate 413. The transparent plate is preferably glass with an anti-reflective coating applied to its surface.

Light remitted by the skin 303 passes within the gun 203 towards a second polarisation filter 415 mounted with the angle of polarisation at ninety degrees to that of the first polarisation filter 412. The second polarisation filter 415 will completely absorb any light reflected from the transparent plate 413 and the skin surface as such reflections will contribute to background noise in the detector. Light remitted from the skin is non-polarised and hence will be polarised by the second polarisation filter 415.

Light passing through the second polarisation filter is focused by a lens 416 onto the CCD detector 417. The type and make of CCD selected depends on factors such as cost and sensitivity required. The type of CCD 417 in the preferred embodiment of this invention is a relatively inexpensive CCD with a relatively narrow dynamic range of sensitivity such as a Sony ICX249AL with a 766×587 pixel array. The effective use of such a CCD is made possible by the method of operation of the apparatus which will be described later.

The intensity of the light remitted by the skin 303 is converted to a charge by the CCD 417. The magnitude of the charge within a pixel of the CCD 417 is dependent on the intensity and duration of the exposure to the remitted light. Charge will accumulate within a pixel of the CCD array corresponding to the intensity of light remitted from a single point of the image.

All CCD arrays have a maximum charge capacity and a sensitivity range. If the illumination is increased beyond this capacity, the excess charge is conducted to earth. This process is known as anti-blooming and prevents the overcharging of one pixel from affecting the charge of an adjacent pixel. The CCD 417 is equipped with an anti-blooming means.

The CCD detector 417 transmits images to the computer 418 which has a digitising card 419. A series of images obtained by the CCD detector 417 can be interpreted by the computer 418, the spectral characteristics calculated and compared to models of normal healthy skin.

The resultant images are presented to an operator in a variety of formats enabling the visualisation of specific spectral features of the remitted light on the display screen 202 which is touch-sensitive to enable the operator to select the images and representations required by selecting icons on the screen by touch.

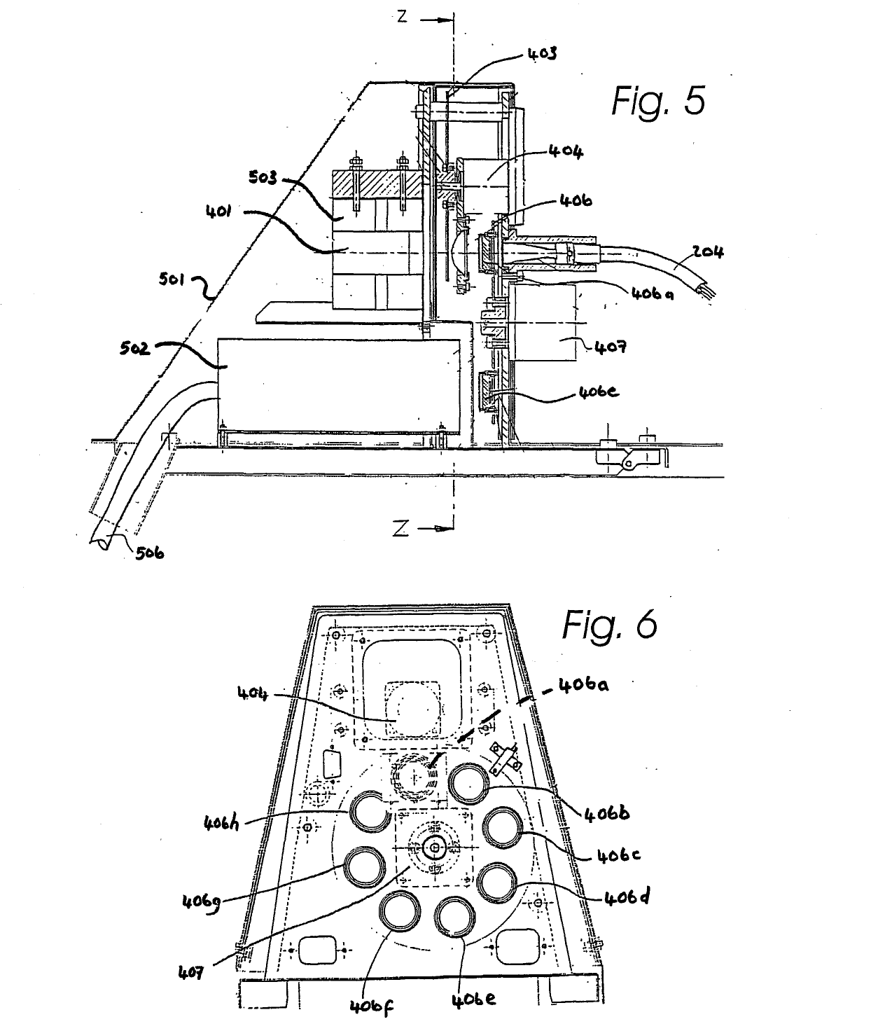

FIGS. 5 and 6

FIGS. 5 and 6 show an actual construction of an apparatus subassembly and components which have been shown schematically in FIG. 4. The same reference numerals are being used to identify equivalent components. The sub-assembly of FIGS. 5 and 6 is contained within a casing 501 which is itself contained within the housing 201. A power pack 502 is connected to a mains power supply by a cable 506. The xenon-arc bulb 401 is provided with a series of heat sinks 503 to prevent overheating. Light emitted by the light source 401 is incident on the shutter disc wheel 403.

Light from the xenon-arc bulb 401 passes through the selected aperture in the shutter disc 403, through the lens 405 and through the selected filter 406a of the filter disc 406, as previously described with reference to FIG. 4. The other filter discs are shown in 406b to 406h respectively. Light leaving the selected filter disc enters the bundle of optical fibres 204 at 504, wherein the illumination is transmitted to the gun 203 (not shown in FIGS. 5 and 6).

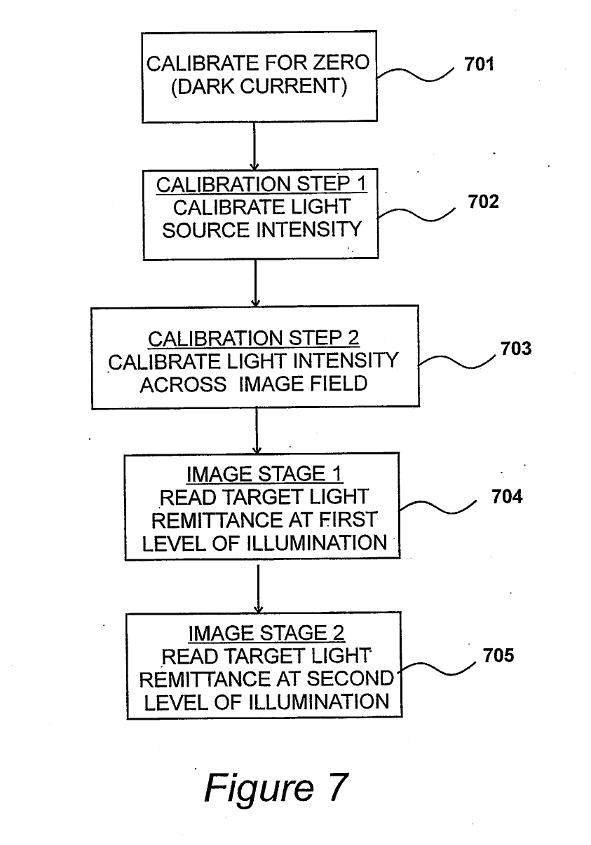

FIG. 7

The operational sequence of the equipment of FIGS. 2 to 6 is shown in FIG. 7.

The initial step 701 is to determine the dark current (or background reading) of the detector so that the zero can be set. This step is followed by a first calibration step 702 which determines the consistency of the intensity of the light emitted by the source over time. There then follows a second calibration step 703 wherein the homogeneity of the intensity of the incident illumination on the desired target area is recorded.

Following the calibration steps 701, 702 and 703, an image is obtained at a first level of illumination (step 704). Finally there is step 705 which incorporates obtaining an image of the target at a second illumination level.

Multiple illumination levels may be incorporated into the operational sequence at stages 702 and 703 and more than two levels may be incorporated in obtaining the image of the target area as in steps 704 and 705. Each of the steps 701 to 705 will now be described in more detail.

The dark current (or background reading) is necessary where the detector produces a residual dark current in the absence of any incident illumination. This is the case when, for example, the detector is a CCD array.

To obtain the dark current an image is obtained from the detector in the absence of any light incident on the detector. The images are recorded and the background intensity calculated across the display. The image analysis software is programmed to subtract the background intensity at each point in an image obtained upon illumination of the detector to correct for the dark current.

In the first calibration step 702, power is supplied to the light source and the intensity of the image remitted from a series of grey patches of known remittance mounted within the image field of the detector is determined. For example, grey patches with defined reflectivities of between zero percent and ninety-five percent may be provided.

The intensity of the light reflected by the calibration patches located within the image field is recorded by the computer 418 and the image analysis software can calculate a correction factor for each image to allow for variations in the intensity of the illumination emitted by the source over time. By this procedure, variations in the illumination intensity incident on the skin, due to variations in the light source, are corrected for which enables the direct comparison of images obtained at different times.

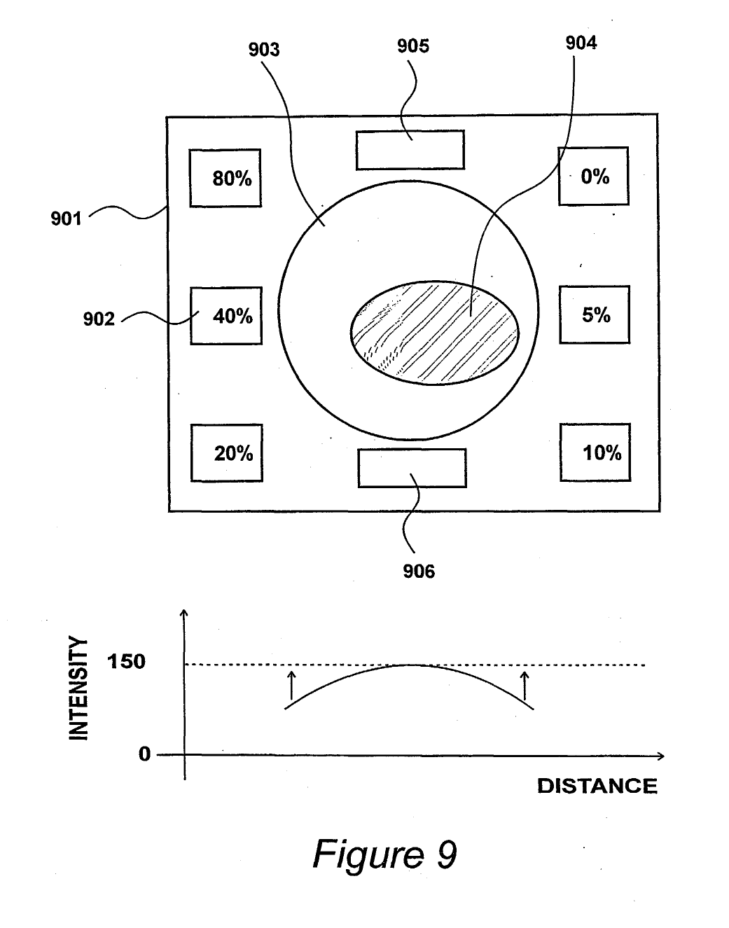

The second calibration step 703, determines the profile of the intensity of the incident illumination across the image field of the detector 417 (see FIG. 9). A homogenous grey surface of known remittance, for example fifty percent remittance, is located adjacent to the external surface of the transparent plate 413 such that incident light, which in use illuminates an area of skin, illuminates the grey surface instead. An image of the light reflected from the homogenous grey surface is obtained by the detector 417. Assuming a homogenous intensity of incident illumination, the intensity of the remitted light will be likewise of homogenous intensity across the homogenous grey surface. Conversely, any inhomogeneities in the intensity of the image are due to corresponding inhomogeneities in the illumination intensity. In a similar manner to the first calibration step, the data from the second calibration step 703 is stored and used to correct images of the skin obtained for inhomogeneities in the intensity of illumination.

The second calibration step is a key feature of the present invention. Following the calibration steps 702 and 703, the next stages involve the imaging of the desired area of the skin. For each of stages 704 and 705, the skin is placed directly in the path of the incident illumination. Preferably the skin surface should be flat and, for this reason, the transparent glass plate 413 is provided which may be pressed against the skin surface to produce a flat area of skin to be imaged. In the operational step 704, the skin is illuminated with light at a first intensity level from the source 401 and an image of light remitted from the skin is obtained via the detector 417. The image is corrected by the image analysis software to account for the dark current 701, variations in the intensity of the light source 702 and inhomogeneities in the illumination across the image 703. Each point of the image detected at a first illumination level has a value of the percentage of light remitted at that point which is derived from the intensity of remitted light detected at the first incident illumination intensity. The spectral composition of the images obtained at a first intensity level of illumination may then be compared with that of an area of normal skin of comparable thickness, either directly by the operator, or by comparing the spectral composition of the remitted light with that of models of the spectral composition within normal healthy skin by the computer software using known methodology.

One of the problems to be overcome by the present invention is the fact that off-the-shelf CCD detectors have a usable dynamic range of sensitivity which is not wide enough to enable good images of widely contrasting targets to be obtained for a given illumination level. For example the presence of a mole in the target area can give rise to a problem because the mole is very dark in comparison with the surrounding skin.

The optimum level of illumination of the surrounding skin will mean that the level of illumination of the mole will not be sufficient to result in a CCD output signal which is greater than the “noise” level of the detector.

This problem is overcome by the present invention, without resorting to the use of more expensive detectors having a greater dynamic range. The solution is to expose the target sequentially to more than one intensity level of illumination, typically two. At the first lower intensity level of illumination the surrounding skin can be imaged but the mole cannot because of inadequate illumination. At the second, higher intensity level of illumination the mole can be imaged but the surrounding skin will be over exposed. By obtaining two or more images at different illumination intensities of light of selected wavelength, a combined image is produced wherein each point of the image displayed is detected within the usable dynamic range of the CCD detector. Therefore, for each point of the image, the appropriate illumination intensity is selected which provides an accurate intensity of remitted light within the usable dynamic range of the detector. The resultant combined image therefore has, at each point, a recorded value of percentage remittance derived from intensity of the selected illumination intensity and the intensity of the remitted light detectable at that selected intensity.

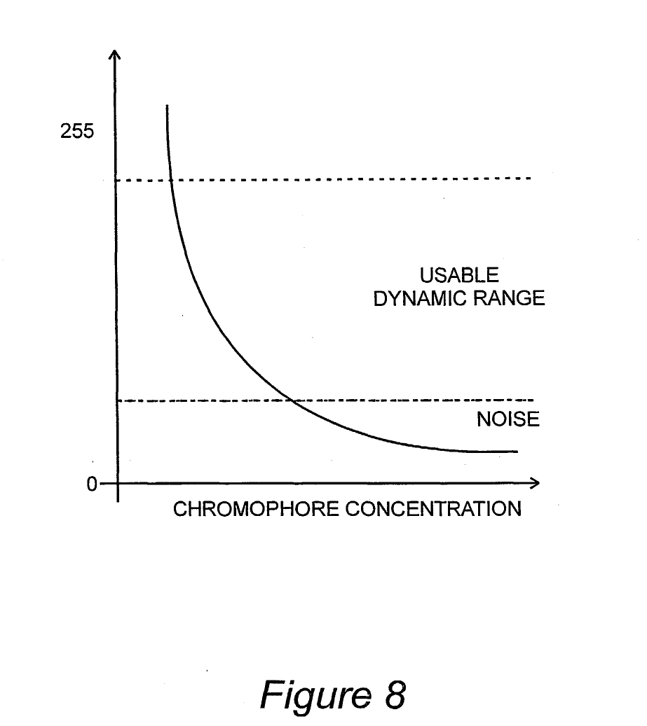

FIG. 8

The dynamic range of a typical CCD detector 417 is shown in FIG. 8. Also shown in FIG. 8 is the effect of the concentration of a chromophore, for example melanin, on the intensity of the light remitted from the skin surface. It should be noted that an increase in concentration of the chromophore results in a decrease in the intensity of remitted light. Consequently, at a specific level of illumination, there will be a lower intensity of remitted light from areas with a high chromophore concentration.

The ratio of the intensity of incident illumination over an area of skin to the amount of remitted light is a constant, which depends on the concentration of specific chromophores within the skin. For this reason, by varying the intensity of illumination of the skin, the intensity of the remitted light from any point of the image may be adjusted to be within the range of the dynamic range of the CCD detector. By dynamic range we mean the range of sensitivity of the CCD, which accounts for background noise and the maximum intensity detectable by the CCD. The dynamic range will depend on the type of CCD and the level of background noise or interference within the image. For example, a typical CCD with an intensity detection range from zero to two hundred and fifty-five may have a dynamic range of fifty to two hundred.

FIG. 9

A schematic representation of the image field of the CCD is shown in FIG. 9. The CCD has an image field 901 which incorporates a series of calibration patches 902. Each patch is a grey surface of known remittance, exemplified in the FIG. 9 as zero percent, five percent, ten percent, twenty percent, forty percent and eighty percent. Also located within the image field is the transparent glass aperture 903 within which a pigmented lesion 904, for example a mole, is located. For a specific illumination intensity, the intensity of the remitted light can be correlated to the light remitted from a one or more calibration patches. This enables the manipulation of the illumination intensity to determine the distribution and concentration of chromophores within areas of the skin of differing remittivity and enable the correlation of the images obtained at multiple illumination intensities.

Mirror patches 905 and 906 may also be incorporated within the image field. Plane polarised light incident on the mirror patches 905 and 906 is reflected without a change in the angle of polarisation. For this reason, the light reflected will be completely absorbed by the second polarisation filter 415 mounted with the angle of polarisation at ninety degrees to the first polarisation filter 412 associated with the ring light 410 (illustrated in FIG. 4). Consequently, in normal operation, the mirror patches 905 and 906 will appear black. The image analysis software is programmed to determine the intensity of the light reflected by the mirror patches 905 and 906. It is required for the correct functioning of the apparatus that the image of the mirror patches 905 and 906 remains black. If for any reason light reflected from the mirror patches 905 and 906 is detected then a problem exists with the polarisation filter arrangement which may give rise to false images and hence, the image will be rejected.

For the purpose of illustration, also shown in FIG. 9 is an intensity calibration across the image (as obtained in step 703, FIG. 7). By virtue of the calibration procedure, such discrepancies in illumination can be accounted for by the image analysis software to enable the interpretation and presentation of the image as if the illumination had been even across the image, as previously described.

Images obtained by the apparatus of the invention are stored within the computer 412 and interpreted using mathematical models as described in, for example, our earlier patent applications WO 98/22023, United Kingdom patent applications 99 12 908 and 99 25 414.

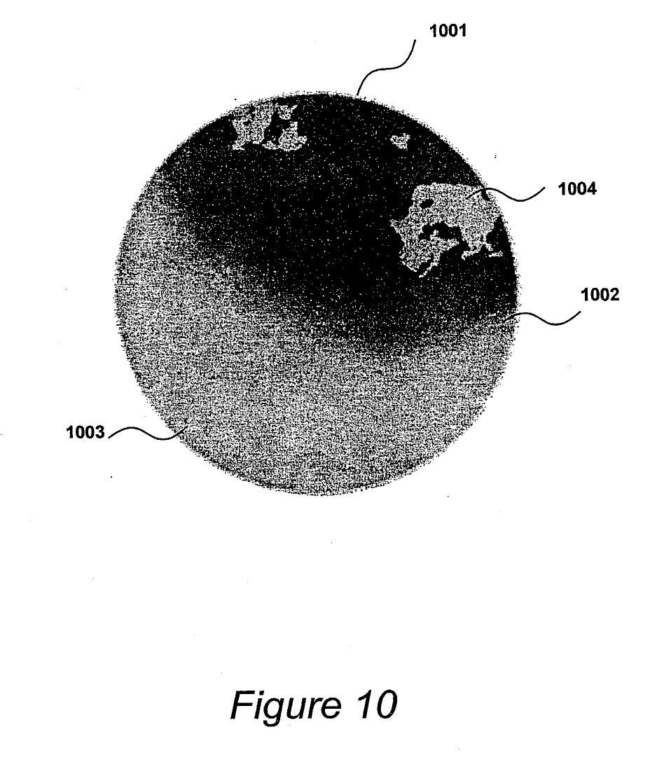

FIG. 10

FIG. 10 shows an example of an image of a malignant melanoma produced by the apparatus of FIGS. 2 to 6 showing an area of skin having a malignant melanoma 1001 with an edge 1002 and an area of non-pigmented skin 1003. Areas of the melanoma where melanocytes have ingressed into the papillary dermis 1004 are overlaid in a contrasting colour. The computer image analysis software can compare the image obtained with that of normal skin of identical thickness. Areas of the image where the distribution differs from that of normal skin can be represented by a suitable colour to enhance the visible features.

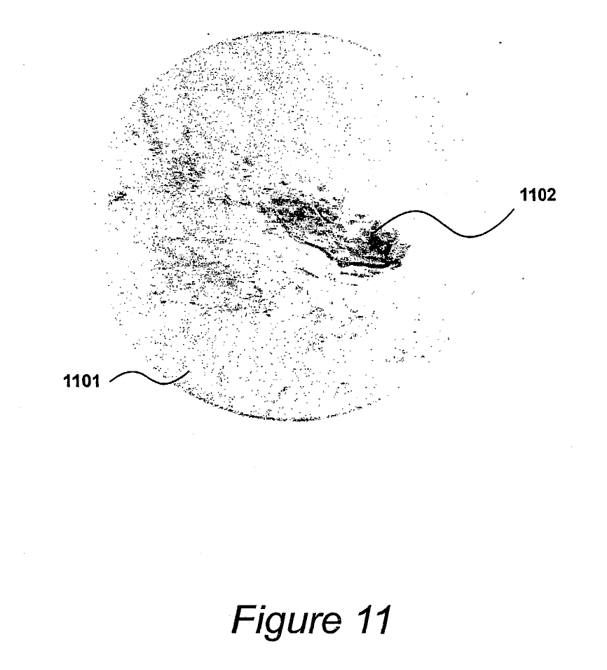

FIG. 11

Similarly, FIG. 11 is an image of a nodular malignant melanoma showing the distribution of haemoglobin. There are clear regions of low concentration of haemoglobin and areas of high concentration where haemoglobin has been concentrated. Differences in the homogeneity of distribution of haemoglobin, for example, are indicative of rapidly enlarging melanoma. Again, the software can compare the distribution of haemoglobin with that of normal skin of identical thickness, using known methodology.

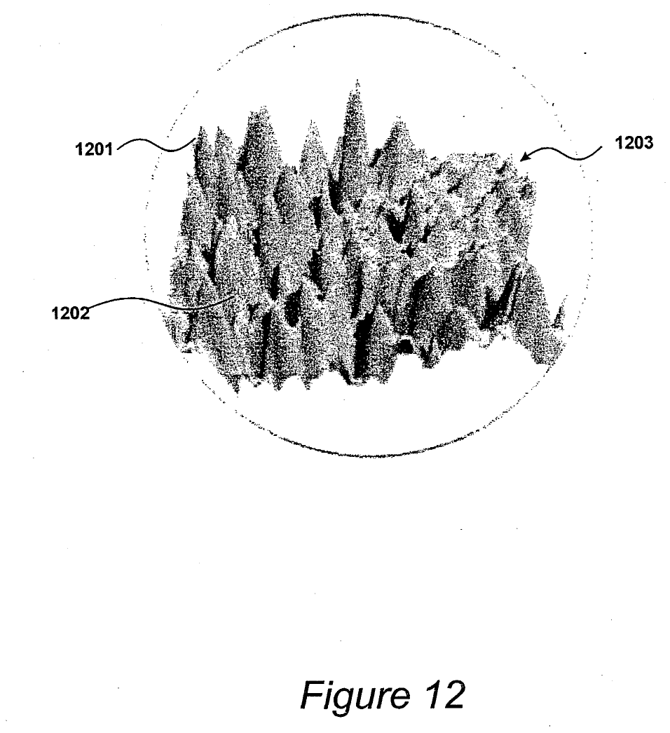

FIG. 12

FIG. 12 shows the profile of the dermal-epidermal junction obtained by the apparatus of the invention. The profile of this junction can be mapped as described in our co-pending United Kingdom patent application number 99 25 414. The peaks 1201 and troughs 1202 of dermal-epidermal junction of normal skin can be seen and an area of flattening 1203 which corresponds to a basal cell carcinoma is also displayed.

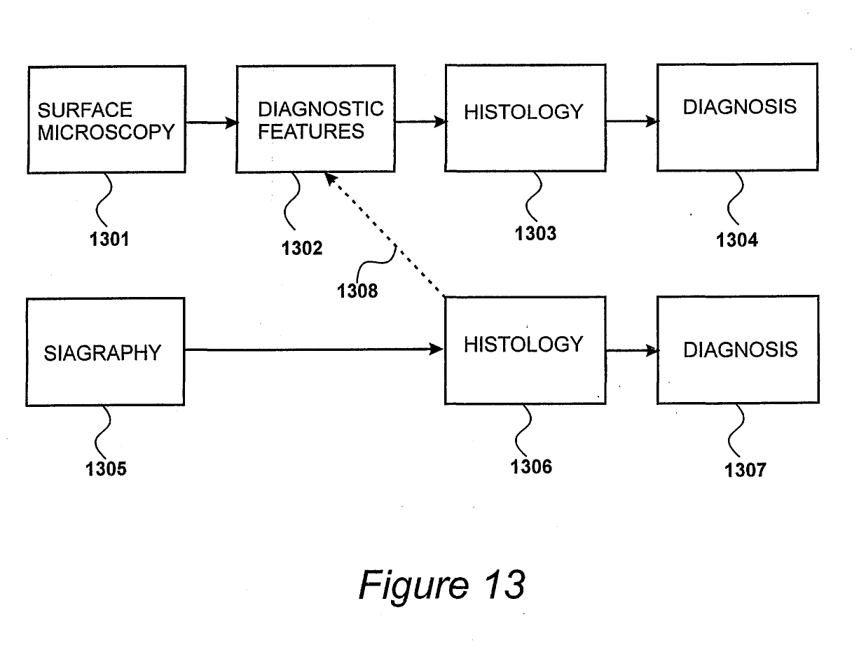

FIG. 13

The conventional approach leading to the diagnosis of skin ailment is shown in the top half of FIG. 13. The skin lesion is examined by surface microscopy 1301 to produce features 1302 which are examined. The recognition of indicative diagnostic features 1302 upon surface examination relies heavily on the observational skills and experience of the clinician. There may then follow a histological examination 1303 by the surgical excision of the lesion and microscopic analysis of the tissue. The combination of information obtained from steps 1302 and 1303 enables the clinician to make a suitable diagnosis at 1305.

The apparatus of the invention enables the provision of images (known by the applicants as SIAGRAPHS) at 1305 which can provide histological information about the skin, without the need for surgical excision.

By this process, histological information 1306 can be provided rapidly and at an earlier stage in the diagnostic procedure 1307 than is the case with the steps 1301 to 1304.

However, in order to provide a clinician with the images/features with which he/she is familiar, the steps of the present invention could include creating the features 1302 from the histological step 1306 (as indicated at 1308).

The apparatus of the invention can be used to obtain a series of images of the skin corresponding to the distribution of various chromophores constituents of the skin. The images obtained by the apparatus of the invention may be correlated with images of the same lesion obtained by macroscopic and microscopic using a standard histological techniques. Specific diagnostic features examined within the images obtained by conventional techniques and can be correlated with the images obtained by the apparatus of the invention. The software may then be programmed to detect and/or enhance specific diagnostic features present.

For example, grey-blue areas are described as representing fibrosis and melanophages in a thickened papillary dermis and constitute an important diagnostic feature for a malignant melanoma with a specificity in the region of ninety-seven percent. The blue-grey areas are identified by reconstructing images obtained by the apparatus of the invention which specifically relate to collagen and dermal melanin. The resultant image accurately correlates with the blue-grey areas identified by standard microscopic and macroscopic analysis techniques.

Similarly, the distribution of specific chromophore within the images of the skin may be selectively combined and enhanced to highlight specific features. Again using the example of a malignant melanoma, an image of the lesion may be overlaid with a highlighted image of a melanin pigment network. The ‘pigment network’ is formed by the varying concentrations of melanin along the undulations of the basal layer of the epidermis which has a ‘honey-combed’ appearance. A highlighted image showing the distribution of epidermal melanin can be selectively overlaid on a macroscopic image of the skin to enable a clinician to view the characteristic pigmented network features.

Further examples of clinical features which can be correlated with the images obtained by the apparatus of the invention include ‘black dots and globules’, ‘multiple blue-grey dots’, ‘radial steaming’, ‘pseudopodia’ and ‘branched streaks’.

In FIGS. 4 to 6, the means by which the skin is illuminated is, in the embodiment described, to select the intensity and wavelength of light from the source prior to the incident light illuminating the skin. Alternatively, the light from the source could be transmitted directly to the skin and the wavelength and intensity of the light remitted from the skin, which is incident on the detector, selected by suitable means.

There are also alternative means by which the intensity light can be selected and furthermore, the intensity may be selected at any point between the light source and the detector. For example, a series of neutral density filters of varying absorptive capacity (which absorb light equally at all incident wavelengths) could be provided that can be selectably orientated into the light path or a means of varying the magnitude of the power supplied to the light source, and hence, the intensity of the emitted light would also suffice.

The selection of the wavelength of light incident can also be selected by alternative means such as by directing the light from onto a diffraction grating or a prism which separate the light into separate wavelengths and the angle of the lens or the diffraction grating or prism adjusted to enable the selection of wavelength constituent required. Alternatively, an electronic adaptive filter could be incorporated into the apparatus of the invention as an electronic means for selecting the wavelength of the light. Again, as for the intensity, the wavelength may be selected at any point between the detector and the light source.

Alternatively, the skin may be illuminated directly by a light source and the detector provided with a means for selecting the intensity and/or the wavelength of the remitted light. For example, the skin may be directly illuminated with a light source at high intensity and the intensity of the remitted light selected by the provision of one or more neutral density filters of varying absorptivity, a shutter disc or alternative means for selecting the intensity. Likewise, the wavelength or wavelength range of light remitted from the skin following illumination with full spectral illumination light incident may be selected by a suitable means, such that the wavelength of remitted light incident on the detector can be selected.

There are also alternative means by which the intensity of the light remitted at specific wavelengths or wavelength ranges can be detected. The detector may be a spectrometer with capacity to scan the intensity of the remitted light over the entire range of visible and infrared wavelengths. In embodiments of the invention where the detector is a spectrometer the means of filtering and selecting the wavelength of light detected is integral within the spectrometer. Alternatively, the detector could be one or more colour cameras, one or more black and white cameras having a coloured filter or, one or more black and white cameras having a coloured light. A cheaper embodiment of the invention could provide a rough estimation of the distribution of chromophores within the skin surface by illuminating the skin with a range of visible and infra-red wavelengths and providing one or more detectors, for example three. One detector could be fitted with a filter which only allows specific infra-red wavelengths to access the camera to provide information on the distribution of collagen and infer the skin thickness. A second detector could be provided with a blue filter and a third provided with a red filter to provide a rough estimation of the distribution of melanin and haemoglobin, for example, within the skin.

Furthermore, the detector may be a single point detector, a single point scanning detector or a multi-point scanning detector which scans the image of remitted light.

In the simplest case, the detector may be an eyepiece or a magnifying glass provided with a filtering means to select specific wavelengths or wavelength ranges through which a suitably trained operator may examine the spectra of light remitted from the skin. Devices are also known by which the infra-red spectral composition of the light may converted into visible light. Such a device could be associated with the eyepiece to enable the operator to view infrared remitted light wavelength, which provide information about the thickness of the skin.

Although the gun 203 is the preferred means by which the light from the source may be transmitted to the skin, there are alternatives that would be suitable. For example, in the simplest embodiment, light from a suitable source can be directed, either physically or manually, onto the desired target skin area without the arrangement of the light pipe described previously. Furthermore, there are alternative methods by which reflections of light from the directly from the source into the detector may be eliminated by, for example, the use of oils on the skin surface to reduce the occurrence of reflections.

The operational sequence may also be modified depending on the detection means. For example, with an expensive CCD array, the dynamic range of intensities detectable is much greater and may enable the illumination of the skin at a high single intensity, such that all the remitted light is within the dynamic range of the detector. This operational procedure would not, however, be suitable for cheaper CCD detectors with more limited dynamic ranges.

It should be emphasised that the apparatus described with reference to FIGS. 4 to 6 is the preferred embodiment of the invention. However, a less than optimum performance may be provided by the alternatives discussed immediately but even such a degraded performance would provide a significant proportion of the benefits of the present invention.

Furthermore, although the preferred embodiment of the invention has been described in reference to the determination of the distribution of chromophores within skin of a human, the technique is equally applicable to the determination of chromophores within any epithelial tissue surface as previously defined.

1. An apparatus configured to create one or more spectral images of an epithelial surface for determination of the concentration and distribution of chromophores within the epithelial tissue surface comprising
a light source to illuminate an area of the epithelial tissue surface with visible and infrared spectral light;
wavelength selection means for selecting the wavelength of light remitted from the epithelial tissue surface;
intensity selection means for selecting the intensity of light remitted from one or more points of the epithelial tissue surface, and
detection means for detecting the intensity of remitted light at one or more points from the epithelial tissue surface to create an image such that each point of the image corresponds to a value of the percentage of incident light remitted.

2. An apparatus as claimed in claim 1, wherein said detection means is a charge coupled display having a usable dynamic range of detection.

3. An apparatus as claimed in claim 2, wherein the intensity of said remitted light at each point of said image is selected such that said intensity is within the dynamic range of the detector.

4. An apparatus as claimed in any preceding claim, wherein said apparatus further comprises a computer having a display screen and image analysis software such that images obtained by the detection means are processed by the image analysis software and displayed on the display screen.

5. An apparatus as claimed in claim 4, wherein the display screen is a touch-sensitive screen enabling the selection of software options by selecting icons on the screen by touch.

6. An apparatus as claimed in any preceding claim, wherein the light source is a xenon-arc bulb.

7. An apparatus as claimed in any preceding claim, wherein the wavelength selection means is selected from the group of wavelength filters, diffraction gratings and prisms.

8. An apparatus as claimed in claim 7, wherein the wavelength selection means is one or more filters mounted about an axis of rotation such that, upon rotation about that axis, each filter can be individually orientated within the light path.

9. An apparatus as claimed in any preceding claim, wherein the intensity selection means for selecting the intensity of the remitted light is selected from the group consisting of apertures of varying dimensions and neutral density filters.

10. An apparatus as claimed in claim 2, wherein the charge-coupled display is fitted with an anti-blooming device.

11. An apparatus as claimed in claim 1, wherein the detection means and wavelength selection means for selecting the wavelength of remitted light is a spectrometer.

12. An apparatus as claimed in any preceding claim, wherein said apparatus is configured to calibrate the intensity of light remitted from the epithelial surface and further comprises:
(i) means to detect the dark current image from the detector;
(ii) one or more calibration patches with surfaces of known remittance mounted within the detective field of the detector such that, upon illumination, the intensity of light reflected by said patches is detected; and
(iii) a surface of defined remittance distribution selectably mountable in the image area of the detector such that, upon illumination, the homogeneity of the incident illumination across the image field can be detected.

13. Apparatus as claimed in claim 12, wherein the calibration patches are one or more grey patches of defined remittance.

14. Apparatus as claimed in claim 12, wherein the surface of defined remittance is a surface of homogenous remittance.

15. A method of determining the concentration and distribution of chromophores within an epithelial tissue using a detector having a defined usable dynamic range, wherein said method comprises the steps of
calibrating the intensity of light source and the homogeneity of the incident illumination
illuminating the epithelial tissue surface at a first intensity of illumination;
detecting the intensity of light remitted from the epithelial tissue surface; and
illuminating the epithelial tissue with at least one further level of illumination and detecting the image such that substantially each point of the image is detected within the dynamic range of the detector.

16. A method according to claim 15, wherein said calibration further comprises the steps of:
obtaining a dark current image from the detector in the absence of illumination;
calibrating the intensity of the light source illumination by detecting the intensity of light reflected from a series of calibration patches, with surfaces of defined remittance, mounted within the detective field of the detector; and
illuminating a surface of defined remittance distribution, obtaining an image and calculating a correction factor for each point of the image to account for inhomogeneities in the illumination intensity of the surface.

17. A method as claimed in claim 15, which further comprises the additional steps of:
correcting said image for inhomogeneities in the illumination intensity the light source; and
correcting the image for variations in intensity across the image.

18. A method as claimed in claim 16, wherein the calibration patches are one or more grey surfaces of defined remittance.

19. A method of creating a combined image having each point of the image detected within the usable dynamic range of the detector wherein said method comprises the steps of:
calibrating the intensity of light source and the homogeneity of the incident illumination;
illuminating the epithelial tissue surface at a first intensity of illumination;
detecting the intensity of light remitted from the epithelial tissue surface; and
illuminating the epithelial tissue with at least one further level of illumination different from said first level
detecting said image and, for each point of said image, selecting said first or second illumination intensity such that substantially each point of the image is detected within the dynamic range of the detector.

20. A method according to claim 19, wherein said calibration further comprises the steps of:
obtaining a dark current image from the detector in the absence of illumination;
calibrating the intensity of the light source illumination by detecting the intensity of light reflected from a series of calibration patches, with surfaces of defined remittance, mounted within the detective field of the detector; and
illuminating a surface of defined remittance distribution, obtaining an image and calculating a correction factor for each point of the image to account for inhomogeneities in the illumination intensity of the surface.

21. A method as claimed in claim 19 or 20, which further comprises the additional steps of:
correcting said image for inhomogeneities in the illumination intensity the light source; and
correcting the image for variations in intensity across the image.
